(19) United States
(12) Patent Application Publication
(10) Pub. No.: US 2015/0265428 A1
(43) Pub. Date: Sep. 24, 2015

(54) ASSIST CONTROL APPARATUS AND METHOD

(71) Applicant: KABUSHIKI KAISHA TOSHIBA, Tokyo (JP)

(72) Inventor: TOSHIKATSU AKIBA, Kisarazu (JP)

(21) Appl. No.: 14/661,356

(22) Filed: Mar. 18, 2015

(30) Foreign Application Priority Data
Mar. 19, 2014 (JP) 2014-056219

(51) Int. Cl. A61F 2/72; G05B 15/02

(52) U.S. Cl. CPC A61F 2/72; G05B 15/02; A61F 2002/701

(57) ABSTRACT

According to one embodiment, an assist control apparatus includes a driving mechanism, an acquisition unit, an estimation unit and a drive unit. The driving mechanism is attached to a leg of a user. The acquisition unit is configured to acquire a status signal indicating a motion of an arm of the user. The estimation unit is configured to determine an assistance timing which is a timing for assisting an action of the user based on changes in the status signal. The drive unit is configured to drive the driving mechanism to generate an assistance power to assist the action of the user in accordance with the assistance timing.

### CROSS-REFERENCE TO RELATED APPLICATIONS

This application is based upon and claims the benefit of priority from Japanese Patent Application No. 2014-056219 filed Mar. 19, 2014, the entire contents of which are incorporated herein by reference.

### FIELD

Embodiments described herein relate generally to an assist control apparatus and method.

### BACKGROUND

In the field of health care, an important issue is to provide an opportunity for elderly people to lead an active life. One of the problems that confront elderly people is difficulty in walking due to a decline in muscular strength, and because of this difficulty, elderly people tend to refrain from going outdoors, and their activity becomes withdrawn. Infrequent exercise may cause weakened muscles, especially for muscles in the legs. Accordingly, it is desirable to provide an apparatus assisting elderly people to walk so that they can walk without difficulty.

An apparatus to assist walking by controlling an actuator by means of a link mechanism, or an apparatus to assist walking by detecting a biosignal according to muscle activity of a user's legs has been developed. In addition, an apparatus assisting a leg motion by estimating the motion by means of a force sensor attached to a user's legs has been developed.

However, since the leg muscles rapidly weaken in comparison to a person's arms, if a biosignal such as a myoelectric potential of the legs is used as a basis for control, the signal used as the basis for control is not stable. In addition, if the apparatus estimates a leg motion by the force sensor attached to the user's legs, the motion of the apparatus may not match the intention or the timing of the user to walk. This may cause uncomfortable assisted walking motions.

### BRIEF DESCRIPTION OF THE DRAWINGS

FIG. 1 is a block diagram illustrating an assist control apparatus according to the first embodiment.

FIG. 2A illustrates a first example of wearing the assist control apparatus.

FIG. 2B illustrates a second example of wearing the assist control apparatus.

FIG. 2C illustrates a third example of wearing the assist control apparatus.

FIG. 2D illustrates a fourth example of wearing the assist control apparatus.

FIG. 2E illustrates a fifth example of wearing the assist control apparatus.

FIG. 2F illustrates a sixth example of wearing the assist control apparatus.

FIG. 2G illustrates a seventh example of wearing the assist control apparatus.

FIG. 3A illustrates an example of wearing a sensor of a status acquisition unit.

FIG. 3B illustrates another example of wearing the sensor of the status acquisition unit.

FIG. 4A illustrates a walk initiation trigger.

FIG. 4B illustrates a walk stoppage trigger.

FIG. 4C illustrates a trigger of standing up.

FIG. 4D illustrates a trigger of sitting down.

FIG. 5 is a flowchart illustrating the operation of the assist control apparatus according to the first embodiment.

FIG. 6 illustrates an example of an assisting operation corresponding to one cycle of a walking motion.

FIG. 7 is a graph illustrating types and timing of assisting operations in a status estimation unit.

FIG. 8 illustrates graphs illustrating the timing of assisting operations when a standard database is used.

FIG. 9 is a block diagram illustrating an assist control apparatus according to the second embodiment.

FIG. 10 is a flowchart illustrating the operation of the assist control apparatus according to the second embodiment.

FIG. 11 is a block diagram illustrating an assist control apparatus according to the third embodiment.

FIG. 12 is a graph illustrating the processing of a delay calculation unit and a correction unit.

FIG. 13 is a block diagram illustrating an assist control apparatus according to the fourth embodiment.

FIG. 14 is a block diagram illustrating an assist control apparatus attached to one arm and one leg according to the fourth embodiment.

FIG. 15 is a graph illustrating an example of a hardware configuration of the assist control apparatus.

### DETAILED DESCRIPTION

In general, according to one embodiment, an assist control apparatus includes a driving mechanism, an acquisition unit, an estimation unit and a drive unit. The driving mechanism is attached to a leg of a user. The acquisition unit is configured to acquire a status signal indicating a motion of an arm of the user. The estimation unit is configured to determine an assistance timing which is a timing for assisting an action of the user based on changes in the status signal. The drive unit is configured to drive the driving mechanism to generate an assistance power to assist the action of the user in accordance with the assistance timing.

In the following, the assist control apparatus and method according to the present embodiment will be described in detail with reference to the drawings. In the embodiment described below, elements specified by the same reference numbers carry out the same operations, and a duplicate description of such elements will be omitted.

### First Embodiment

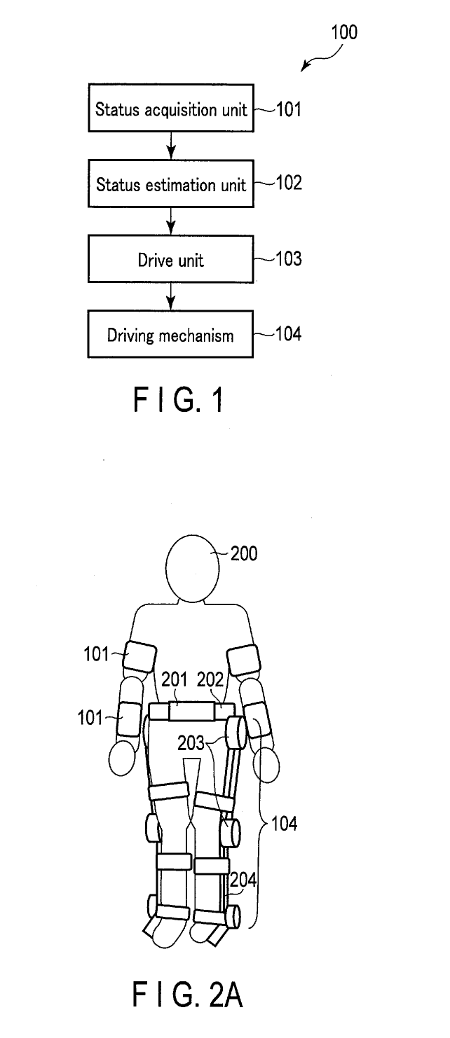

The assist control apparatus according to the first embodiment will be explained with reference to FIG. 1.

The assist control apparatus 100 according to the first embodiment includes a status acquisition unit 101, a status estimation unit 102, a drive unit 103, and a driving mechanism 104.

The status acquisition unit 101 acquires a status signal indicating a motion of a user's arm. A status signal is a biosignal including the myoelectric potential measured by a myoelectric potential sensor attached to an upper part of the user's arm, a sensor signal of a position or an angle of the arm measured by an attitude sensor attached to an upper part of the user's arm, or a sensor signal regarding the speed (or the angle speed) and acceleration (or the angle acceleration) of the motion of the arm measured by an acceleration sensor. The status acquisition unit 101 may acquire time sequence data of signal values as a status signal, and the signal values include an arm myoelectric potential value, an arm acceleration value, and an angle indicating an arm's position and direction. The myoelectric potential is assumed to be a surface muscle myoelectric potential, but may be a myoelectric potential of inner muscle.

The status estimation unit 102 receives a status signal from the status acquisition unit 101, determines a timing for generating assistance power (or referred to as an assistance timing) in accordance with the sequential change of the status signal, and generates timing information. The assistance power is a power to assist a user's motion, and is assumed to have a predetermined strength in this embodiment. A timing corresponding to the value of a reference pattern that is equal to a signal value of the status signal acquired at the status acquisition unit 101 may be determined as the assistance timing by referring to the reference pattern. The reference pattern is a pattern of sequential data of status signals that have been acquired beforehand. For example, the situation where a user walks will be considered below. It is assumed that the speed of moving a right arm and a left arm is represented by a sine wave, where the forward movement is positive, and the backward movement is negative. In this case, if the speed of the right arm is changed from a negative value to a positive value, assistance to move a left leg forward will be made.

When an assistance power is added to a specific motion such as a user's walking, the assistance timing may be determined as described above. However, when an assistance power is added to various motions of the user, the status estimation unit 102 may estimate the user's motion in accordance with the sequential change of the status signal. After estimating the user's motion, the status estimation unit 102 may determine an assistance timing to assist the user's motion, and generate timing information including information regarding the user's motion and the assistance timing. The user's motions in this embodiment include a motion of the user's walk (walking motion), a motion of the user trying to sit down (sitting-down motion), and a motion of the user trying to stand up (standing-up motion). The user's motion may be estimated by associating the pattern of sequential data of status signals (the reference pattern) with predetermined motions that the user may make, and selecting a motion corresponding to the reference pattern that is closest to a pattern of the sequential data of the status signal acquired at the status acquisition unit 101. The assistance timing may also be determined by referring to the reference pattern corresponding to the estimated user's motion.

The drive unit 103 receives the timing information from the status estimation unit 102, and generates a control signal to drive the driving mechanism 104 so that an assistance power is made at the assistance timing indicated by the timing information. When information regarding the user's motion is included in the timing information, a control signal to drive the driving mechanism 104 according to the user's motion and the assistance timing is generated.

The driving mechanism 104 includes a motor to be attached to a leg of the user (for example, waist, knee and ankle) and generating torque. The driving mechanism 104 is driven with a driving power generating an assistance power to the user upon reception of the control signal from the drive unit 103. For the walking motion, the driving mechanism 104 may be driven to generate an assistance power to assist the user to step forward and to support the user's body. For the sitting-down motion, the driving mechanism 104 may be driven to generate an assistance power to support the user's weight when sitting down. For the standing-up motion, the driving mechanism 104 may be driven to generate an assistance power to support the user's weight when standing up. The driving mechanism 104 may be an aid which has a general assistance function to deliver an assistance power to the legs, and be controlled by the drive unit 103.

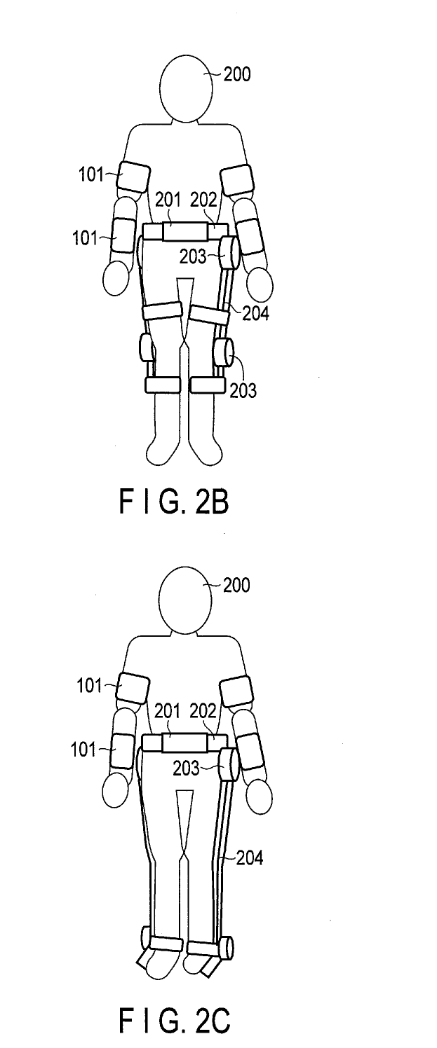

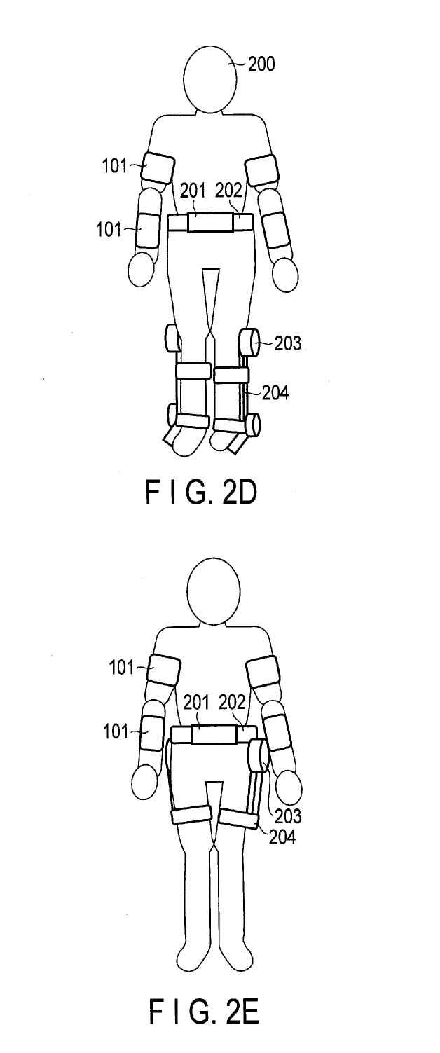

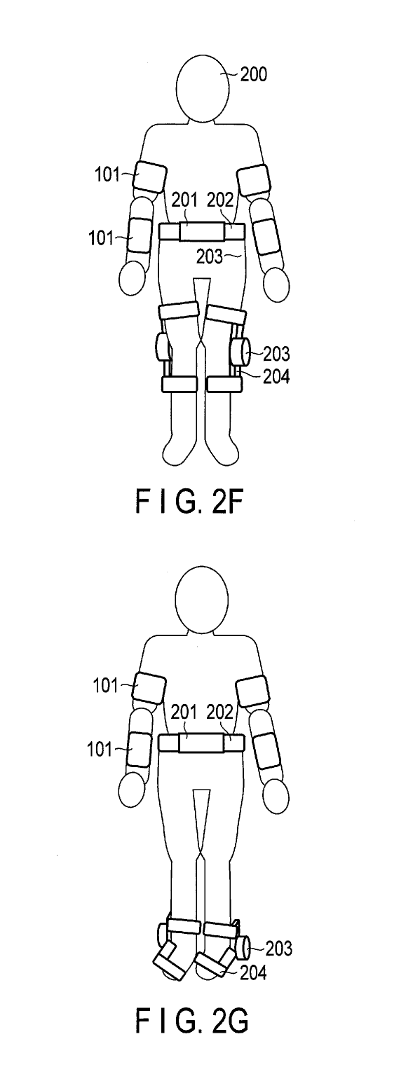

An example of wearing the assist control apparatus 100 will be explained with reference to FIGS. 2A to 2G.

As shown in FIG. 2A, the status acquisition unit 101 is attached to the upper parts and front parts of the arms of a user 200. A case 201 comprising a control circuit including the status estimation unit 102, the drive unit 103, and a power supply unit supplying power to the assist control apparatus 100 is fixed to the waist of the user 200 by means of a holding means 202. The driving mechanism 104 is attached to the legs of the user 200.

The driving mechanism 104 includes a driving source 203 and a linking mechanism 204. The driving source 203 is a motor, for example, and is linked with the linking mechanism 204. The linking mechanism 204 extends along the leg of the user, and is fixed at a knee or a thigh. In response to an instruction from the drive unit 103, the driving source 203 generates torque. If the torque is delivered to the linking mechanism 204 linked with the driving source 203, the torque is applied to the user 200 as an assistance power. The driving source 203 rotates the linking mechanism 204 to apply the assistance power to the user. Accordingly, it is desirable that the driving source 203 is attached to the waist, knee, and ankle.

The driving mechanism 104 is not limited to extend from the waist to the knee to the ankle of the user 200, as shown in FIG. 2A, but may be attached as shown in FIGS. 2B to 2G.

In FIG. 2B, the driving source 203 is attached to each of the waist and the knee of the user 200, and the linking mechanism 204 is attached to link the driving sources 203 at the waist and the knee. FIG. 2B is an example of assisting leg movement below the knee with the knee as a support point. In FIG. 2C, the driving source 203 is attached to each of the waist and the ankle of the user 200, and the linking mechanism 204 is attached to link the driving sources 203 at the waist and the ankle. FIG. 2C is an example of assisting movement around the hip joint and ankle. In FIG. 2D, the driving source 203 is attached to each of the knee and the ankle of the user 200, and the linking mechanism 204 is attached to link the driving sources 203 at the knee and the ankle. In FIG. 2E, the driving source 203 is attached only to the waist, and the linking mechanism 204 is attached to extend from the waist to the thigh. FIG. 2E is an example of assisting movement centered around the hip joint. In FIG. 2F, the driving source 203 is attached only to the knee to assist rotation around the knee. In FIG. 2G, the driving source 203 is attached only to the ankle, and the linking mechanism 204 is attached to extend from the ankle to the toe. FIG. 2G is an example of assisting movement around the ankle.

As shown in FIGS. 2B to 2G, the assistance power can be applied only to a body part where muscle is weakened, and is not applied to parts having healthy muscle strength. This prevents an excessive deterioration in muscle strength due to applying an assistance power to healthy parts.

As shown in FIGS. 2D, 2F and 2G, when the driving mechanism 104 (driving source 203 and linking mechanism 204) is separated from the case 201 including the control circuit, a control signal may be wirelessly transmitted to the driving mechanism 104.

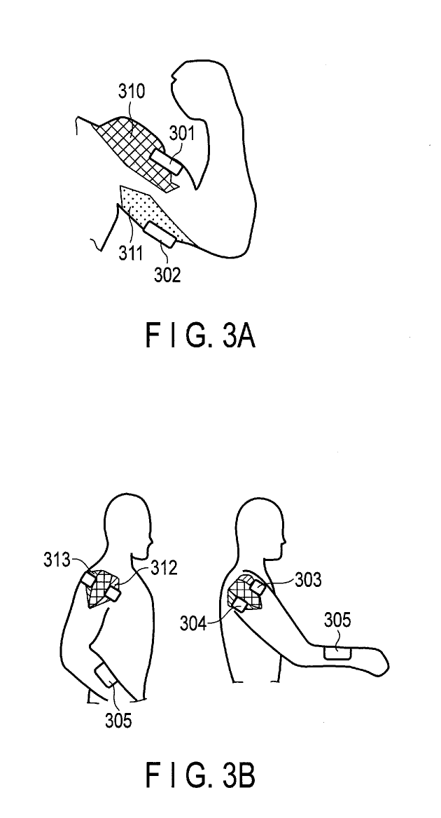

An example of wearing the status acquisition unit 101 to acquire a status signal will be explained with reference to FIG. 3.

In FIG. 3A, a biceps myoelectric potential sensor 301 to measure the myoelectric potential of a biceps muscle 310, and a triceps myoelectric potential sensor 302 to measure the myoelectric potential of myoelectric potential sensor 301 to measure the myoelectric potential of a triceps muscle 311 are attached to an upper arm of the user. The biceps myoelectric potential sensor 301 and the triceps myoelectric potential sensor 302 are provided as the status acquisition unit 101. In general, when a person walks, the biceps are tensed if swinging the arm forward, and the triceps are tensed if swinging the arm backward. The status acquisition unit 101 acquires sequential data as a status signal of myoelectric potential for the motion of swinging an arm when walking.

As shown in FIG. 3B, a forward part myoelectric potential sensor 303 to measure the myoelectric potential of a forward part of a deltoid muscle 312 may be attached to the forward part of the upper arm, and a rear part myoelectric potential sensor 304 to measure the myoelectric potential of a rear part of deltoid muscle 313 may be attached to the rear part of the upper arm. When the forward part myoelectric potential sensor 303 and the backward part myoelectric potential sensor 304 are used, the myoelectric potential value may be acquired as a status signal in a manner similar to the case where the biceps myoelectric potential sensor 301 and the triceps myoelectric potential sensor 302 are used.

The status estimation unit 102 may increase the assistance power in accordance with the myoelectric potential value. For example, if the user wishes to increase the assistance power when walking, the user can swing the arms strongly by using the biceps. If the myoelectric potential values measured at the biceps myoelectric potential sensor 301 and the triceps myoelectric potential sensor 302 are greater than the case of ordinary walking, the status estimation unit 102 instructs the drive unit 103 to increase the assistance power.

For another example of increasing the assistance power, a forearm myoelectric potential sensor 305 to measure the myoelectric potential of forearm muscles (musculus extensor digitorum, for example) may be attached. In this case, the user can tense the forearm to increase the assistance power. The status estimation unit 102 detects a trigger to increase the assistance power based on the change of the myoelectric potential value measured from the forearm myoelectric potential sensor 305, and instructs the drive unit 103 to increase the assistance power upon the detection.

The aforementioned status signal is the myoelectric potential value acquired by the myoelectric potential sensor; however, the status signal may be the values acquired by the acceleration sensor or the attitude sensor. For example, when using the acceleration sensor, arm swing acceleration is acquired. In this case, the status estimation unit 102 may instruct the drive unit 103 to increase the assistance power if the acquired acceleration is greater than a threshold. When using the attitude sensor, the angle the arm is swung is acquired. In this case, the status estimation unit 102 may instruct the drive unit 103 to increase the assistance power if the maximum angle of the swung arm is greater than a threshold.

The status estimation unit 102 may decrease the assistance power in accordance with the myoelectric potential value. For example, if the user's arm swing is weaker than a usual walking motion, the status estimation unit 102 may determine it as a trigger to decrease the assistance power, and instruct the drive unit 103 to decrease the assistance power.

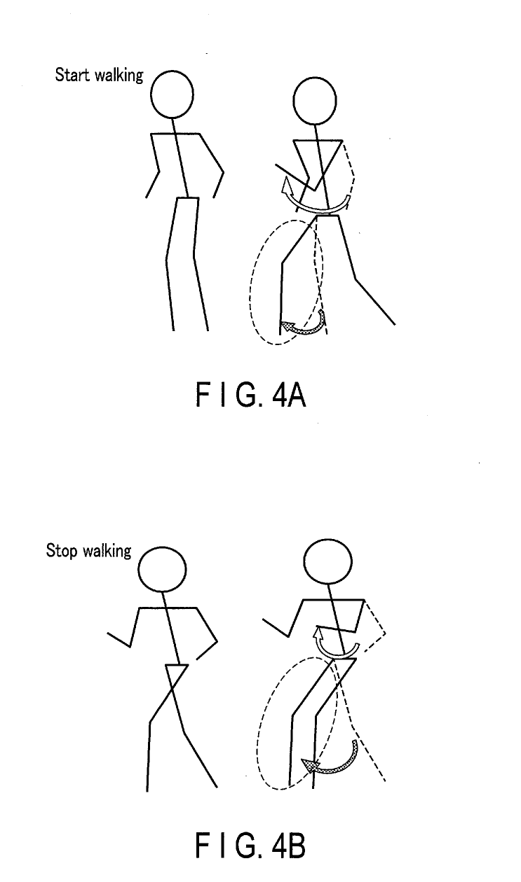

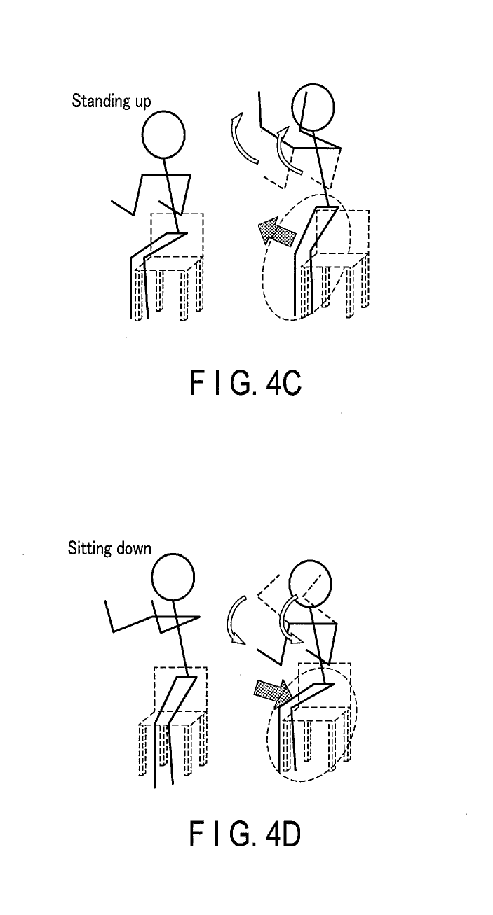

Next, the relationships between arm motion and walking, sitting-down, and standing-up motions will be explained with reference to FIGS. 4A to 4D.

FIG. 4A shows a motion of trying to start walking. From the status of standing upright, as the right arm is swung forward, the left leg moves. As the left arm is swung forward, the right leg moves. The action of swinging an arm forward is set as a trigger for starting walking. The action of alternately swinging the arms forward is regarded as a walking motion.

FIG. 4A shows a motion of stopping walking. For example, from the status where the right arm is swung forward, and the left leg is stepped forward, the left arm is swung forward and stopped at the position symmetrical to the right arm to stand upright. The action of stopping both arms is set as a trigger for stopping walking.

FIG. 4C shows a motion of standing up from a chair 401. When standing up, the motion of the legs is similar to the arm motion of moving from the forward position to the upward position. Thus, the motion of moving the arms from the forward position to the upward position when sitting is set as a trigger for standing up.

FIG. 4D shows a motion of sitting on the chair 401. When sitting down, the motion of the legs is similar to the motion of the arms moving from the forward position to the downward position. Thus, the motion of moving the arms from the forward position to the downward position is set as a trigger for sitting down.

The status estimation unit 102 may estimate the motion of the user based on the positions and motions of the user's body and arms as shown in FIGS. 4A to 4D.

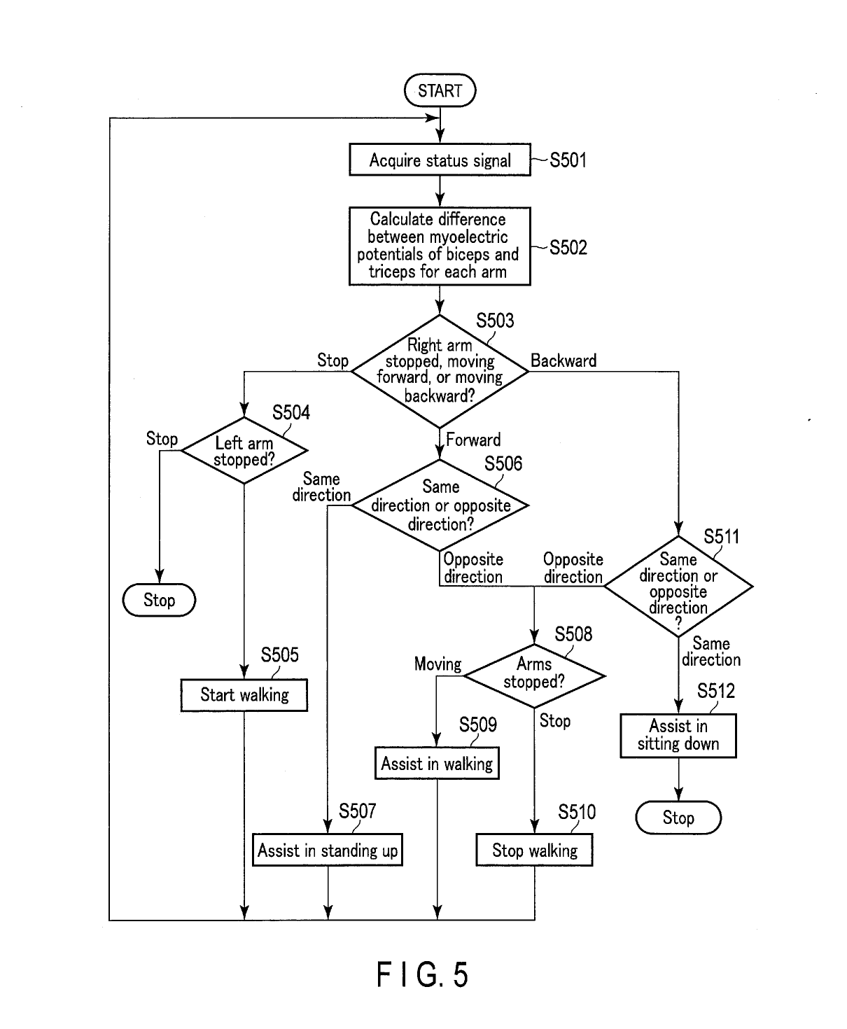

Next, the operation of the assist control apparatus 100 according to the first embodiment will be explained with reference to the flowchart shown in FIG. 5.

In step S501, the status acquisition unit 101 acquires a status signal from the upper parts of the user's arms. It is assumed that the myoelectric potential of the biceps and the myoelectric potential of the triceps are acquired at predetermined sampling intervals as status signals.

In step S502, the status estimation unit 102 calculates the difference between the myoelectric potentials of the biceps and triceps for each arm.

In step S503, the status estimation unit 102 determines whether the right arm is stopped, moving forward, or moving backward. If the right arm is stopped, step S504 is executed. If the right arm is moving forward, step S506 is executed. If the right arm is moving backward, step S511 is executed.

The myoelectric potential of the biceps when the user swings an arm forward will be greater than the myoelectric potential of the triceps of the same arm. The status estimation unit 102 compares the myoelectric potentials of the biceps and triceps, and determines that the arm is moved forward if the myoelectric potential of the biceps is greater than that of the triceps. When the user swings an arm backward, the triceps may be stressed. Accordingly, the status estimation unit 102 determines that the arm is moved backward if the myoelectric potential of the triceps is greater than that of the biceps. If the myoelectric potential is zero or stable, the status estimation unit 102 determines that the arm is stopped.

However, since the myoelectric potential varies for each person, if a database is created in which data of myoelectric potential when an arm is moved forward and myoelectric potential when an arm is moved backward are associated with each other, an accurate determination can be realized by referring to the database.

In step S504, the status estimation unit 102 determines whether or not the left arm is stopped. If the left arm is stopped, it is assumed that the user stops walking, and the processing is terminated. If the left arm is not stopped, step S505 is executed.

In step S505, the status estimation unit 102 determines that the user starts walking based on the detection that the left arm is moving while the right arm is stopped. Then, the processing returns to step S501, and the same processing is repeated.

In step S506, the status estimation unit 102 determines whether or not the left arm is moving in the same direction as the right arm or in a direction opposite to the right arm. If the left arm is moving in the same direction as the right arm, i.e., a forward direction, step S507 is executed, and if the left arm is moving in the direction opposite to the right arm, i.e., a backward direction, step S508 is executed.

In step S507, the status estimation unit 102 estimates that the user is trying to stand up based on the detection that both arms are moving forward, which is a trigger of standing up. The status estimation unit 102 determines the timing when the user is standing up as an assistance timing. The timing when the user is standing up may be directly after the trigger of standing up is detected. The drive unit 103 receives timing information regarding the user's standing up, and generates a control signal to drive the driving mechanism 104 at the timing when the user is standing up. The driving mechanism 104 generates a driving power to assist the user's motion of standing up based on the control signal to apply the assistance power to the user. Then, the processing returns to step S501, and the same processing is repeated to a status signal subsequently sampled.

In step S508, the status estimation unit 102 determines whether or not the right and left arms are stopped. If the arms are not stopped, step S509 is executed, and if the arms are stopped, step S510 is executed.

In step S509, the status estimation unit 102 estimates that the user is walking based on detecting that both arms are continuously swung forward and backward, and determines that the timing when a leg is stepped forward is set as an assistance timing. The drive unit 103 receives timing information regarding the user's stepping forward, and generates a control signal to drive the driving mechanism 104 at the timing when the user is stepping forward. The driving mechanism 104 generates a driving power based on the control signal. Then, the processing returns to step S501, and the same processing is repeated to a status signal subsequently sampled.

In step S510, the status estimation unit 102 estimates that the user is stopped based on the detection that both arms are stopped from the status where the arms are swung forward and backward. Then, the processing returns to step S501, and the same processing is repeated to a status signal subsequently sampled.

In step S511, the status estimation unit 102 determines whether the left arm is moving in the same direction as the right arm or in the direction opposite to the right arm. If the left arm is moving in the same direction as the right arm, i.e., the backward direction, step S512 is executed, and if the left arm is moving in the direction opposite to the right arm, i.e., the forward direction, step S508 is executed.

In step S512, the status estimation unit 102 estimates that the user is trying to sit down based on the detection that both arms are moving backward, which is a trigger of sitting down. The status estimation unit 102 determines the timing when the user is sitting down as an assistance timing. The timing when the user is sitting down may be directly after the trigger of sitting down is detected. The drive unit 103 receives timing information regarding the user's sitting down, and generates a control signal to drive the driving mechanism 104 at the timing when the user is sitting down. The driving mechanism 104 generates a driving power to assist the user's motion of sitting down based on the control signal to apply the assistance power to the user. Then, the processing returns to step S501, and the same processing is repeated to a status signal subsequently sampled. The operation of the assist control apparatus 100 according to the first embodiment is completed by the above steps.

The assistance power to be applied for the user's action such as a standing-up motion in step S507, a walking motion in step S509, and a sitting-down motion in step S512 may increase or decrease in accordance with the strength of the user's arm swing.

For example, for the user's standing-up motion and walking motion, the assistance power may increase when the measured myoelectric potential is equal to or greater than a threshold. When the user is standing up, the user strongly moves the arms upward from the forward position. In this case, if the myoelectric potential is equal to or greater than the threshold, the status estimation unit 102 instructs the drive unit 103 to increase the assistance power in comparison with the predetermined strength. When the user starts walking, since the myoelectric potential increases by the user strongly swinging an arm, if the myoelectric potential is equal to or greater than the threshold, the status estimation unit 102 instructs the drive unit 103 to increase the assistance power.

In step S501, the status signal is assumed to be a biosignal regarding the myoelectric potential. However, the direction of arm movement can be also calculated by the angle of the arm sensed by the attitude sensor and the acceleration of the arm sensed by the acceleration sensor. For example, in step S502, the status estimation unit 102 determines whether an arm is swung forward or backward by acquiring sequential data of an arm angle in the gravity direction sensed by the attitude sensor attached to an arm. If the acquired sequential data shows a small change, the status estimation unit 102 determines that the arm is stopped.

In addition, the arm swing is estimated based on sequential data of arm acceleration sensed by the acceleration sensor. If the acceleration shows zero, the status estimation unit 102 determines that the arm is stopped. Accordingly, the arm motion and direction can be estimated by using the arm angle sensed by the attitude sensor and the arm acceleration sensed by the acceleration sensor, in a way similar to the case of using the myoelectric potential.

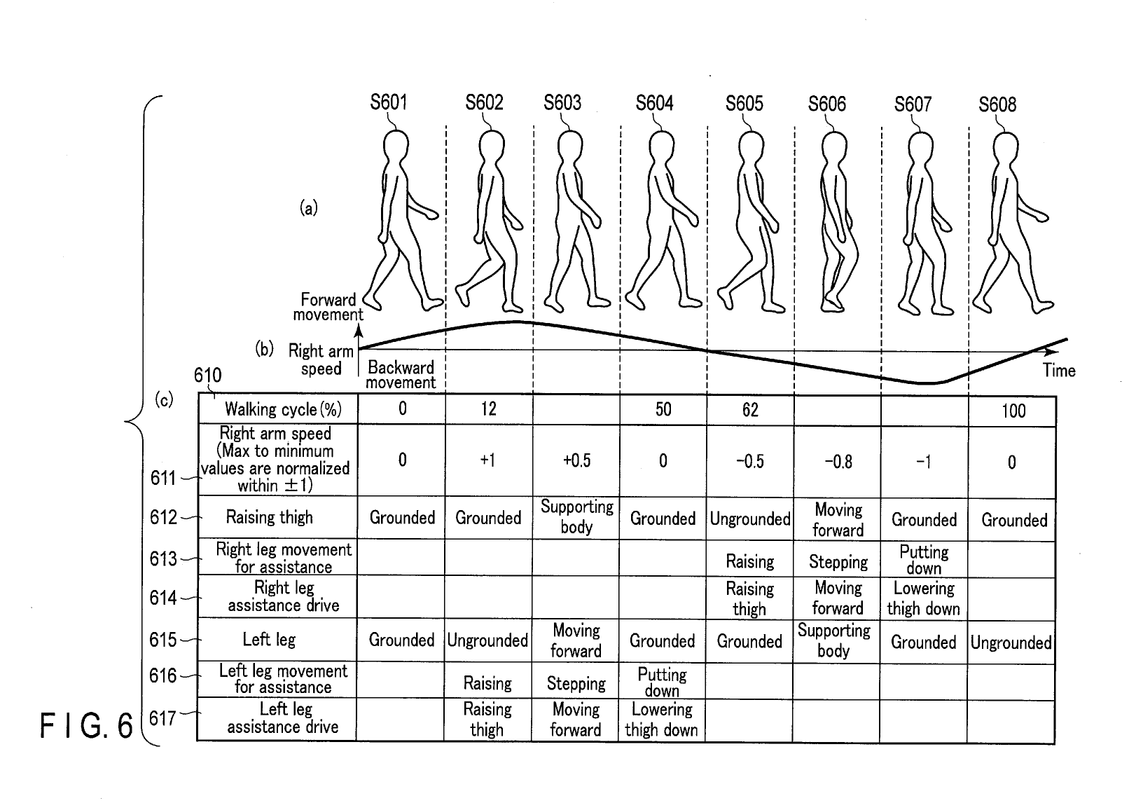

Next, examples of assistance operations corresponding to one cycle of a walking action will be explained with reference to FIG. 6.

FIG. 6 (a) is a schematic diagram representing a user's walking motion, FIG. 6 (b) is a graph showing the relations between the direction, the speed, and time that the right arm is swung, and FIG. 6 (c) is a table showing the relationship between leg movement (right and left legs) relative to the speed of the right arm and the driving status of the assist control apparatus 100. In this embodiment, we focus on the movement of the right arm, and do not mention the movement of the left arm.

FIG. 6 (c) shows a walking cycle 610 from step S601 to step S608. A walking cycle is completed at step S608, and the walking cycle will reach 100% in step S608.

A right arm speed 611 is a normalized value where the maximum speed of moving forward is 1, and the maximum speed of moving backward is −1 (i.e., the minimum speed based on the forward motion).

A right leg 612 shows the status of the right leg. The status includes grounded (touching the ground), supporting the body, moving forward, and ungrounded (not touching the ground).

A right leg movement for assistance 613 indicates how the right leg moves. The right leg movement for assistance 613 includes raising, stepping, and putting down.

A right leg assistance drive 614 indicates what kind of assistance power is applied to the right leg by the driving mechanism 104 to the user. The right leg assistance drive 614 includes raising a thigh, moving forward, and Lowering the thigh down.

A left leg 615 shows the status of the right leg. The status includes grounded, supporting the body, moving forward, and ungrounded, the same as the right leg 612. A left leg movement for assistance 616 indicates how the right leg moves, such as raising, stepping, putting down, the same as the right leg movement for assistance 613.

A left leg assistance drive 617 indicates what kind of assistance power is applied to the left leg by the driving mechanism 104 to the user, such as raising the thigh, moving forward, lowering the thigh down, similar to the right leg assistance drive 614.

In step S601, the right leg of the user is placed forward, and the left leg is placed backward. This status is a walking initiation status. In step S601, the right arm speed is zero, and the right and left legs are grounded.

In step S602 and step S603, the user swings the right arm and the left leg leaves the ground. At the same time, the body is supported by the right leg, and the left leg steps forward. In this case, the right arm speed shows the maximum speed.

In step S604, the forward movement of the left leg is completed, and the forward movement of the right arm is completed. In this case, the right arm speed is zero.

In steps S605 to S608, as the right arm moves backward, the right leg starts moving forward, leaves the ground, and is stepped forward while the left leg supports the body. At the time when the forward movement of the right leg is completed, and the backward movement of the right arm is completed, the positions of the user's legs and arms become the same as those in step S601. A walking cycle is then completed.

The status estimation unit 102 stores the table regarding the walking sequence as shown in FIG. 6 (c) as a database.

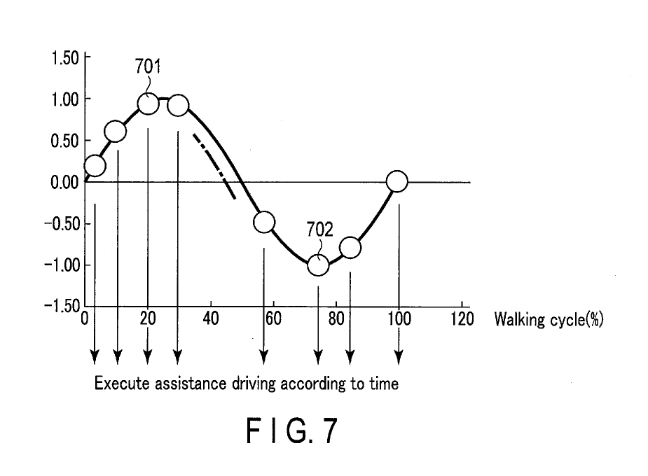

An example of determining a timing and a type of assisting operation in a status estimation unit 102 will be explained with reference to FIG. 7.

The graph shown in FIG. 7 indicates changes of status signals where acceleration of the right arm is used as a status signal. The vertical axis shows the amplitudes of status signals which are normalized so that the maximum value is 1, and the minimum value is −1. The horizontal axis shows a walking cycle as shown in FIG. 6. The walking cycle includes the time duration from when a right leg is stepped forward, to when the left leg is stepped forward, and until the right leg is stepped forward again. The time when walking is initiated is represented as 0%, and the time when a cycle of walking is completed is represented as 100%.

The status estimation unit 102 acquires a graph as shown in FIG. 7 by the sequential data of status signals acquired at the status acquisition unit 101 when the user is walking. The cycle of the graph of FIG. 7 corresponds to a walking cycle, and also corresponds to the right arm speed shown in FIG. 6 (b). The timing when an assistance power is needed in the walking sequence can be determined by calculating a corresponding point between the sequential data of status signals and the right arm speed shown in FIG. 6 (b) and referring to the database regarding the walking sequence as shown in FIG. 6 (c).

For example, timing 701, in which the status signal shows a maximum value, corresponds to the timing in which the right arm speed shown in FIG. 6 (b) is maximum, which is step S602 in FIG. 6 (a). The status estimation unit 102 outputs an instruction signal to the drive unit 103 to perform “drive raising the thigh” in the left leg assistance drive 617 in FIG. 6 (c) by referring to the database. The drive unit 103 drives the driving mechanism 104 to raise the thigh upon reception of the instruction signal. For example, timing 702 in which the status signal shows a minimum value corresponds to the timing in which the right arm speed shown in FIG. 6 (b) is minimum, which is step S607 in FIG. 6 (a). The status estimation unit 102 outputs an instruction signal to the drive unit 103 to perform “drive lowering the thigh” in the right leg assistance drive 614 in FIG. 6 (c) by referring to the database. The drive unit 103 drives the driving mechanism 104 to lower the thigh upon reception of the instruction signal. In FIG. 7, the acceleration is used as a status signal; however, the speed of arm movement calculated by the myoelectric potential may be used as a status signal.

The table shown in FIG. 6 (c) may be prepared by measuring the user's walking beforehand; however, a database of a standard walking cycle may be used.

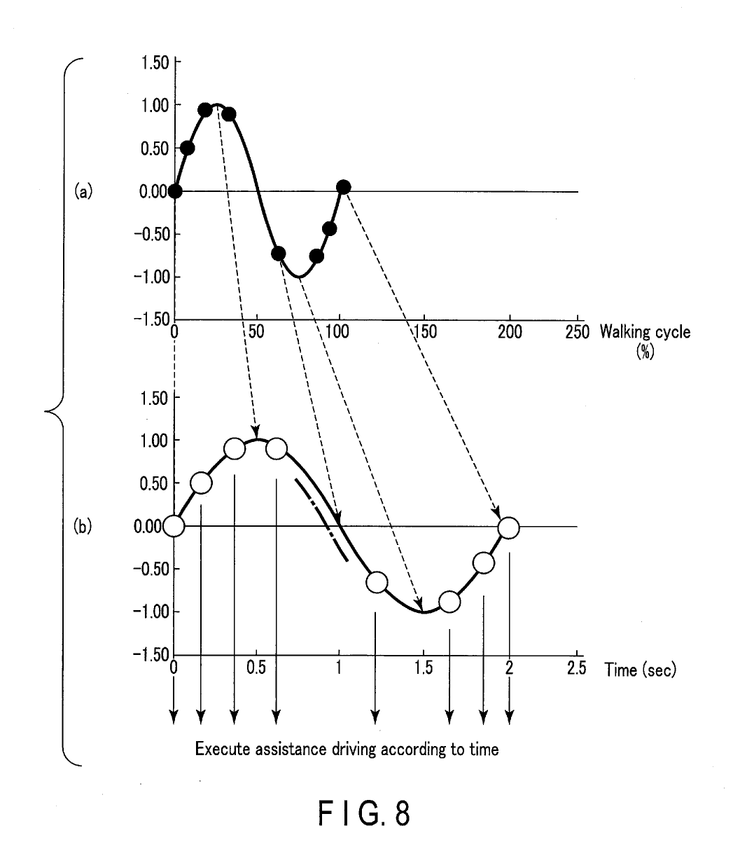

An example of determining a timing and a type of assisting operation when the standard database is used will be explained with reference to FIG. 8.

FIG. 8 (a) is a graph showing the arm speed in a standard walking cycle included in the standard database. FIG. 8 (b) is a graph obtained by converting the standard database to the user's walking cycle.

In the standard database, the relations between the time and the arm speed within a walking cycle (starting with 0% and ending with 100%) are recorded, and the arm speed and timing for an assistance operation are associated with each other.

For the user's walking, the sequential change of status signals acquired at the status acquisition unit 101 is measured. If a walking cycle takes 2 seconds, the standard database is converted as shown in FIG. 8 (b). By referring to the standard database, and assuming that at the time when a walking cycle is completed is 100%, the timings for assistance operations to the user can be calculated based on the ratio of the walking cycle of the standard database (100%) to the walking cycle of the user (2 seconds). In addition, based on the maximum and minimum values of arm swing speed of the user, the numeral values in the vertical axis can be associated with the standard database. FIG. 8 (b) shows an example where the walking cycle of FIG. 8 (a) is converted to the user's walking cycle. FIGS. 8 (a) and (b) show that the user's walking speed is slower than the standard database.

If the timings, status signals (arm speed in this example), and assistance operations by the drive unit 103 and the driving mechanism 104 are associated with each other in the walking cycle of the standard database, the status estimation unit 102 may determine the assistance operation and the timing based on the value of status signal acquired from the status acquisition unit 101 by referring to the standard database.

According to the first embodiment, the user's motion can be estimated based on the status signals acquired by the arm's motion, and the assistance power is applied to the user through the driving mechanism at the timing of walking, standing up, or sitting down in accordance with the estimated user's motion. Accordingly, it is possible to reliably and appropriately assist the user's motion. At the same time, the user does not feel discomfort when receiving assistance.

Furthermore, by referring to the change in status signals of the user, the assistance timing can be determined based only on the relationship between the elapsed walking time and the walking cycle of the standard database to assist walking of the user.

### Second Embodiment

In the first embodiment, it is assumed that a predetermined power level is applied as an assistance power. However, it is possible to apply an assistance power more naturally by changing the strength during the overall action. In addition, since muscle strength varies depending on the user, the predetermined strength may be excessive or insufficient for a particular user. Accordingly, in the second embodiment, the strength of assistance power to be applied (amount of assistance) is calculated in accordance with the motion or timing of motion of the user. Calculating the necessary strength of assistance power allows the assist control apparatus to apply a suitable assistance power in accordance with the user's motion.

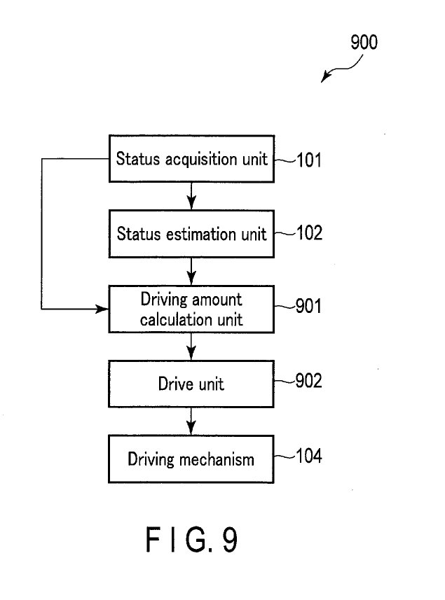

The assist control apparatus according to the second embodiment will be explained with reference to the block diagram of FIG. 9.

The assist control apparatus 900 according to the second embodiment includes the status acquisition unit 101, the status estimation unit 102, the driving mechanism 104, a driving amount calculation unit 901, and a drive unit 902.

The status acquisition unit 101, the status estimation unit 102, and the driving mechanism 104 perform the same operations as those in the first embodiment, and the explanations thereof will be omitted.

The driving amount calculation unit 901 receives a status signal from the status acquisition unit 101, and information regarding the estimated user's motion and timing information from the status estimation unit 102. The driving amount calculation unit 901 calculates an assistance amount of assistance power in accordance with the user's motion and the timing based on the status signal.

The drive unit 902 receives the information regarding the user's motion, the timing information and the amount of assistance from the driving amount calculating unit 901, and generates a control signal to drive the driving mechanism 104 so that an assistance power of the amount of assistance is made at the timing indicated by the timing information.

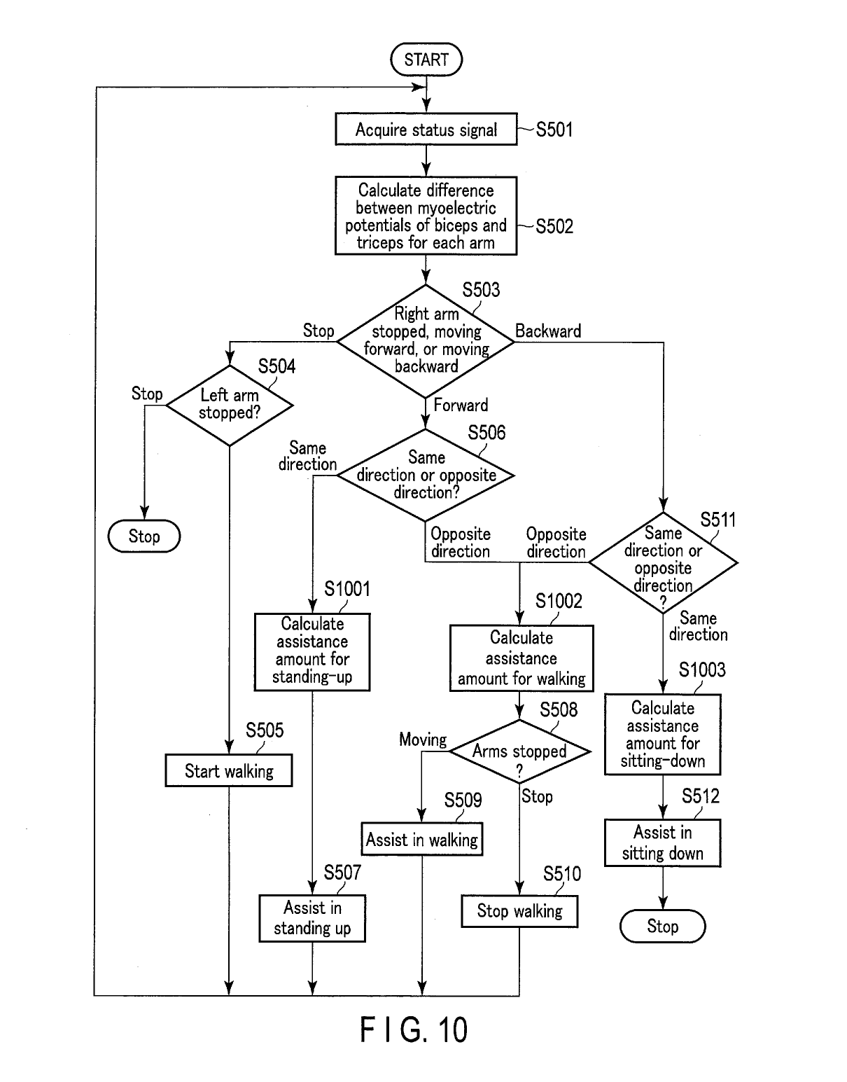

Next, the operation of the assist control apparatus 900 according to the second embodiment will be explained with reference to the flowchart shown in FIG. 10.

Steps S501 to S512 are the same as those in the first embodiment, and the explanations thereof will be omitted.

In step S1001, the driving amount calculation unit 901 calculates an amount of assistance power for the standing-up motion. The assistance amount may vary in accordance with a predetermined time interval, for example. The initial value of the amount of assistance may be large, and the value may decrease in accordance with the progress of the motion of standing-up.

In step S1002, the driving amount calculation unit 901 calculates an amount of assistance power for the walking motion. For example, in the table shown in FIG. 6(c), the amounts of assistance for right leg assistance and left leg assistance are set beforehand, and the set assistance amounts may be used in the timings corresponding to the value of the status signals.

In step S1003, the driving amount calculation unit 901 calculates an amount of assistance power for the sitting-down motion. The initial value of the amount of assistance set for the sitting-down motion may be large, and the value may decrease in accordance with the progress of the sitting-down motion.

According to the second embodiment, the amount of assistance, which is the strength of assistance power, is calculated in accordance with the user's motion and timing of the motion, and a suitable amount of assistance power is applied to the user. This realizes applying a suitable assistance power to the user more naturally.

### Third Embodiment

If the decline of the user's legs is advanced, when the user is trying to walk, the legs cannot move in synchronization with the movement of the arms, and the leg movement may be delayed from the arm movement. In addition, when exercising the legs, it is important to forcedly synchronize the movements of the legs and the arms.

For the above situations, the third embodiment corrects the difference between the movements of the legs (lower limb) and the arms (upper limb) in consideration of the delay of movement of the legs. Correcting the difference allows the assist control apparatus to apply a suitable assistance power to the user.

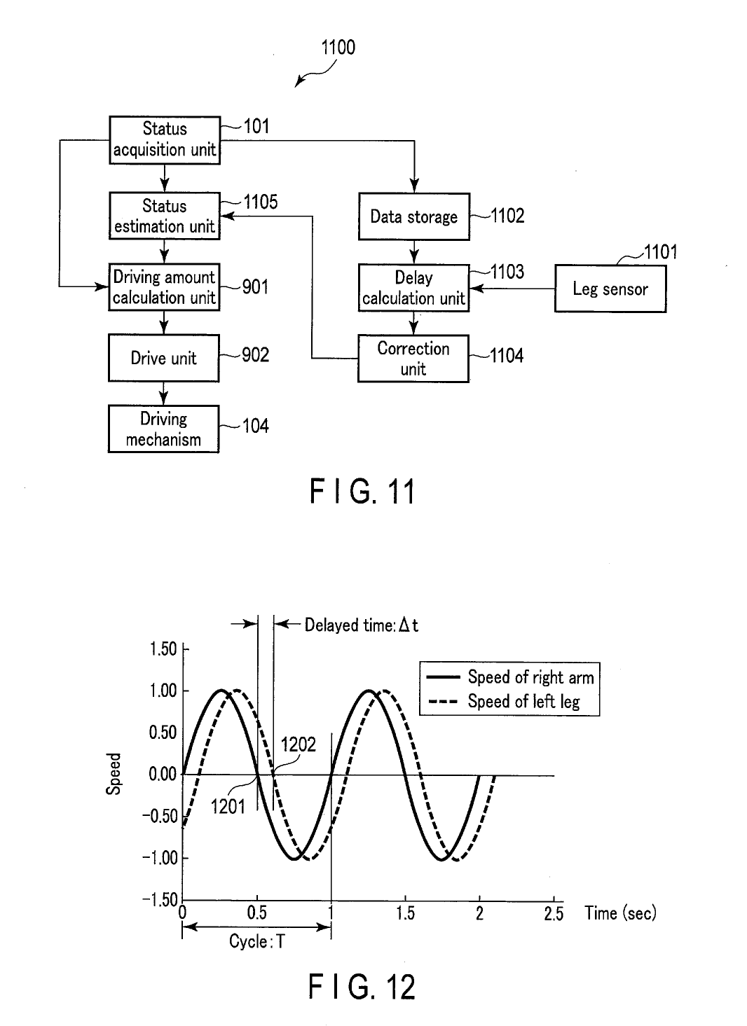

The assist control apparatus according to the third embodiment will be explained with reference to the block diagram of FIG. 11.

The assist control apparatus 1100 according to the third embodiment includes the status acquisition unit 101, the driving mechanism 104, the driving amount calculating unit 901, the drive unit 902, a leg sensor 1101, a data storage 1102, a delay calculation unit 1103, a correction unit 1104, and a status estimation unit 1105.

The status acquisition unit 101, the driving mechanism 104, the driving amount calculating unit 901, and the drive unit 902 perform the same operations as those in the first and second embodiments, and the explanations thereof will be omitted.

The leg sensor 1101 is a rotation sensor and a strength sensor connected to the driving mechanism 104. The leg sensor 1101 measures a waist or knee rotation angle and strength, and obtains the measured value as a detection signal.

The data storage 1102 receives and stores sequential arm speed data as a status signal from the status acquisition unit 101. The data storage 1102 may store sequential data for one walking cycle.

The delay calculation unit 1103 receives the sequential data of arm speed from the data storage 1102, and the sensor value from the leg sensor 1101, and calculates a delayed time of leg movement relative to the arm movement.

The correction unit 1104 receives the delayed time from the delay calculation unit 1103, and generates a correction instruction to correct the difference indicated by the delayed time. Specifically, the correction unit 1104 may generate an instruction to advance the timing by the delayed time.

The status estimation unit 1105 receives the correction instruction from the correction unit 1104, and generates timing information indicating an updated timing which is advanced by the delayed time it. The other operations of the status estimation unit 1105 are the same as those of the status estimation unit 102 of the first embodiment.

Next, the operations of the delay calculation unit 1103 and the correction unit 1104 will be explained with reference to FIG. 12.

FIG. 12 shows the relationship between the speeds and walking cycles of the right arm and left leg. The vertical axis represents the speed that is normalized so that the maximum value is 1, and the minimum value is −1. The horizontal axis represents time. In the third embodiment, one walking cycle of the user is completed in one second. In FIG. 12, the solid line indicates the speed of the right arm, and the broken line indicates the speed of the left leg. The leg sensor 1101 is assumed to be attached to the left knee.

The knee rotation angle speed and the speed of the leg become zero at almost the same timing in walking. When the knee rotation angle speed is zero, the speed of the leg becomes zero.

The arm speed may be calculated from status signals. The delay calculation unit 1103 calculates the difference between the time when the arm speed becomes zero and the time when the leg speed becomes zero to obtain the delayed time of the leg movement relative to the arm movement. For example, in FIG. 12, the differential time Δt between time 1201 when the speed of the right arm becomes zero and time 1202 when the speed of the left leg becomes zero is the delayed time of the leg movement relative to the arm movement.

The correction unit 1104 converts the duration of time for one walking cycle shown in the horizontal axis of FIG. 12 (i.e., 1 second) into a percentage based on the database shown in FIG. 6 (c) so that the timing of assistance in the database is converted in accordance with the user's walking cycle. The delayed time Δt is also converted in the same rate. Based on the results, the correction unit 1104 generates a correction instruction to select an updated timing in which each timing is advanced by the delayed time Δt, based on the data of the previous walking cycle. The status estimation unit 1105 receives the correction instruction, and generates timing information indicating a updated timing which is advanced by the delayed time Δt. The drive unit 902 and the driving mechanism 104, which are post-processing formats of the status estimation unit 1105, drive an assistance operation in accordance with the selected timing to correct the time delay. Since the walking motion is a repetition of simple movements, the tendencies of the user's walking motion can be predicted by correcting the movement using the past data.

The data storage 1102 may store data of the initial one walking cycle to use it repeatedly, or update the data of one walking cycle to store the latest data.

The user may add a suitable condition instead of the delayed time Δt. Accordingly, the correction unit 1104 may increase or decrease the calculated delayed time Δt. In addition, the optimal assistance power may vary depending on the user. Thus, the correction unit 1104 may generate a correction instruction to the drive unit 103 to modify a control factor between a driving signal and a driving power so that the driving power can be adjusted in response to the user's instruction.

There may be a case where the timing is shifted due to mechanical reasons because of conditions where the driving mechanism 104 is used, instead of due to human factors concerning the condition of the user. That is, there may be a case where the timing of the status estimation unit 1105 is shifted from the timing of driving of the driving mechanism 104 (also referred to as driving timing). In this case, if the difference between the timing of the status estimation unit 1105 and the driving timing of the driving mechanism 104 is equal to or greater than a predetermined value, the difference may be adjusted to fall within the predetermined value. Specifically, the drive unit 902 receives a detection signal including the strength of driving power generated at the driving mechanism 104 acquired at the leg sensor 1101 and the time when the driving power is generated. The drive unit 902 performs calculation using a control gain, or the like, so that the driving mechanism 104 is driven at a suitable driving power in accordance with the timing estimated by the status estimation unit 1105.

According to the third embodiment, the delayed time of the leg movement relative to the arm movement is calculated, and an assistance power is generated at the timing in which the delayed time has been corrected. This enables the application of an assistance power to the user at a more suitable timing.

### Fourth Embodiment

The aforementioned embodiments assume the case where a status detection unit and a driving mechanism are attached to both arms and legs. In contrast, the fourth embodiment assumes the case where a status detection unit is attached to one of the arms, or the case where a status detection unit is attached to one of the arms and a driving mechanism is attached to one of the legs. The attachment example of the fourth embodiment can reduce the weight and the cost of the apparatus.

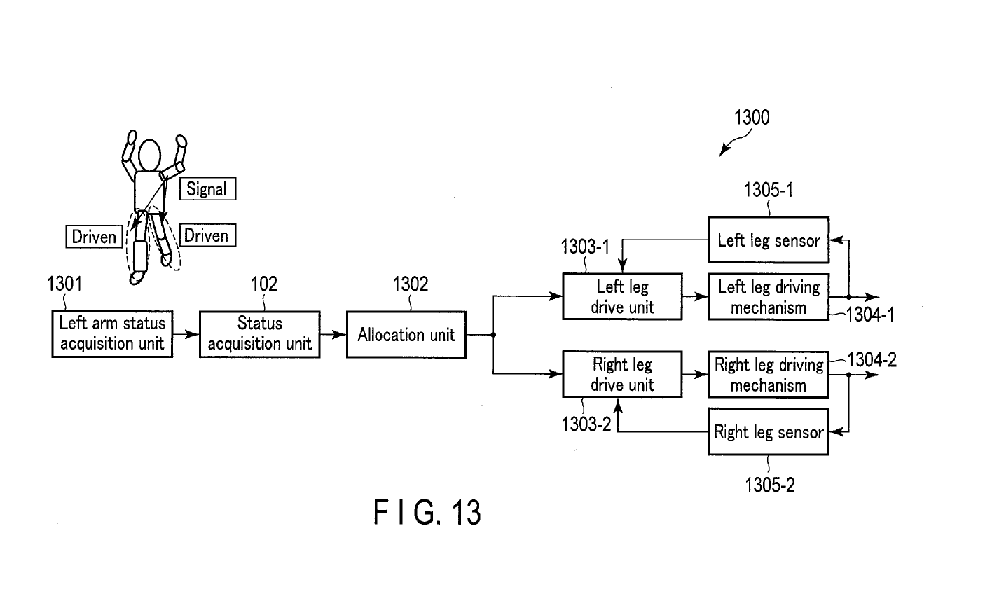

The assist control apparatus according to the fourth embodiment will be explained with reference to the block diagram of FIG. 13. In this embodiment, it is assumed that a status acquisition unit is attached to the left arm of the user; however, it can be attached to the right arm of the user.

The assist control apparatus 1300 according to the fourth embodiment includes a left arm status acquisition unit 1301, the status acquisition unit 102, an allocation unit 1302, a left leg driving unit 1303-1, a right leg driving unit 1303-2, a left leg driving mechanism 1304-1, a right leg driving mechanism 1304-2, a left leg sensor 1305-1, and a right leg sensor 1305-2.

The operation of the left arm status acquisition unit 1301 is similar to that of the status acquisition unit 101. The operation of the status estimation unit 102 is similar to that stated in the aforementioned embodiments. The operations of the left leg driving unit 1303-1 and the right leg driving unit 1303-2 are similar to those of the drive unit 103. The operations of the left leg driving mechanism 1304-1 and the right leg driving mechanism 1304-2 are similar to those of the driving mechanism 104. The operations of the left leg sensor 1305-1 and the right leg sensor 1305-2 are similar to those of the leg sensor 1101. Accordingly, the explanations of these members will be omitted.

The allocation unit 1302 receives an instruction signal from the status estimation unit 102, determines which of the left leg driving mechanism 1304-1 and the right leg driving mechanism 1304-2 is to be driven at the timing of applying an assistance power, and sends a control signal to the selected driving mechanism. The right and left arms alternately move in a similar way when walking. Thus, the allocation unit 1302 may generate an instruction signal to alternately add an assistance power to the right and left legs based on a status signal of the arm to which the status acquisition unit 101 is attached. That is, if the left arm moves forward, an instruction signal to add an assistance power to the right leg is generated, and if the left arm moves backward, an instruction signal to add an assistance power to the left leg is generated.

Since the status acquisition unit 101 is attached to one of the arms, triggers for assuming initiation of walking, stopping walking, standing-up, and sitting-down should be different from those in the aforementioned embodiments. For example, a trigger of standing-up is a motion of raising an arm to which the left arm status acquisition unit 1301 is attached in the direction orthogonal to the direction of swinging an arm in the walking motion. A trigger of sitting-down is a motion of putting an arm to which the left arm status acquisition unit 1301 is attached down in the direction orthogonal to the direction of swinging an arm in the walking motion. A trigger of stopping walking is stopping an arm to which the status acquisition unit 101 is attached regardless of the walking motion. That is, a movement different from the arm swing during walking is set as a trigger of stopping walking. The status estimation unit 102 may estimate the motion of the user by detecting a trigger based on the positional relationship between the user's body and an arm.

In a case where the user has hemiparesis, an assistance power may be applied to one side of the body suffering from hemiparesis based on the movement of an arm which can move.

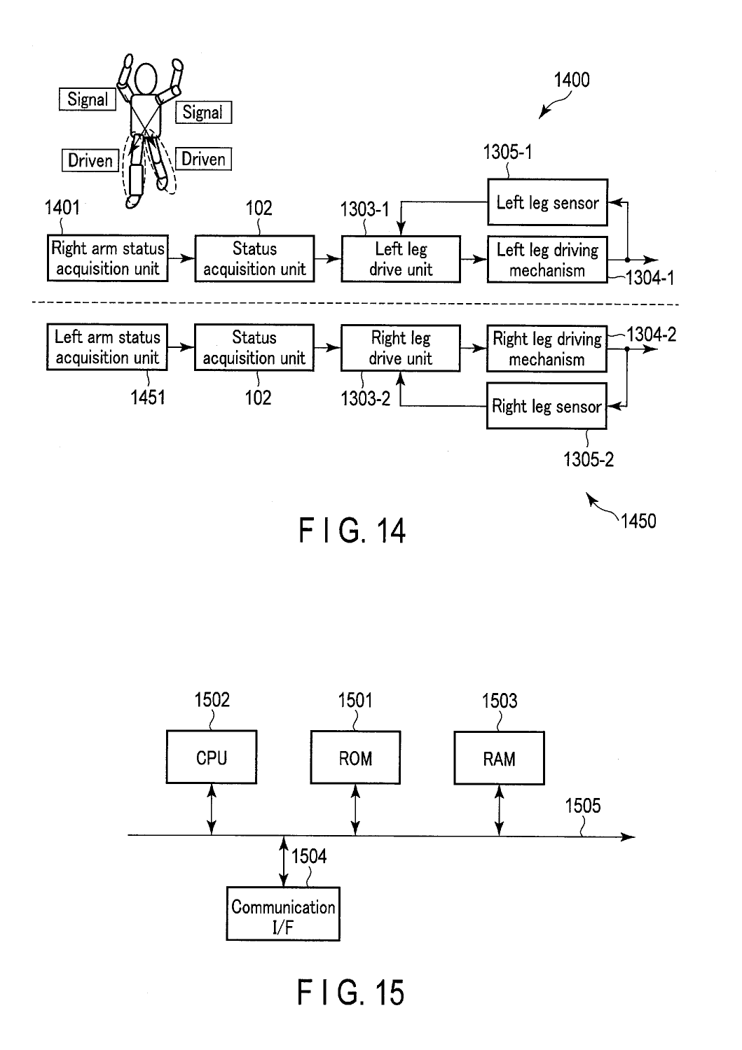

An example of the operation of the assist control apparatus where the user attaches the status acquisition unit 101 to an arm, and attaches the driving mechanism 104 to a leg will be explained with reference to the block diagram of FIG. 14.

The assist control apparatus 1400 shown in FIG. 14 is for driving the left leg based on the movement of the right arm. The assist control apparatus 1400 includes a right arm status acquisition unit 1401, the status estimation unit 102, the left leg driving unit 1303-1, the left leg driving mechanism 1304-1, and the left leg sensor 1305-1. The user attaches the right arm status acquisition unit 1401 to the right arm, and the left leg driving mechanism 1304-1 to the left leg.

The assist control apparatus 1450 is for driving the right leg based on the movement of the left arm. The assist control apparatus 1450 includes a left arm status acquisition unit 1451, the status estimation unit 102, the right leg driving unit 1303-2, the right leg driving mechanism 1304-2, and the right leg sensor 1305-2. The user attaches the left arm status acquisition unit 1451 to the left arm, and attaches the right leg driving mechanism 1304-2 to the right leg.

The operation of members included in the assist control apparatuses 1450 and 1400 are similar to those in the assist control apparatus 1300 shown in FIG. 13.

FIG. 14 shows an example where the left leg driving mechanism is controlled by the movement of the right arm, and the right leg driving mechanism is controlled by the movement of the left arm. However, the left leg driving mechanism may be controlled by the movement of the left arm, and the right leg driving mechanism may be controlled by the movement of the right arm.

According to the fourth embodiment, an assistance power can be applied to both legs or one of the legs by the movement of one of the arms that is able to move. Thus, the assist control apparatus can add a suitable assistance power even when the user is only able to move one of their arms. Also, the fourth embodiment is able to achieve reductions in the weight and cost of the apparatus.

FIG. 5 shows the hardware configuration of the assist control apparatus according to the aforementioned embodiments.

The assist control apparatus includes a ROM 1501 storing an assist control program to execute the operations of the status acquisition unit 101, the status estimation unit 102, and the drive unit 103; a CPU 1502 controlling each component included in the assist control apparatus in accordance with the program stored in the ROM 1501; a RAM 1503 storing data such as a reference pattern and a reference database required for controlling the assist control apparatus; an I/F 1504 communicating through the network; and a bus 1505 connecting each component.

The assist control program may be stored in a computer-readable storage medium such as a CD-ROM, flexible disk (FD), or DVD as an installable or executable format.

In this case, the assist control program is read from the storage medium to be run, and loaded on a main storage of the assist control apparatus 100, thereby implementing the functions of each component shown in FIG. 15 in software on the main storage.

In addition, the assist control program may be stored in a computer connected to the network through the Internet, and downloaded through the network and the I/F 1504 to be executed.

The flow charts of the embodiments illustrate methods and systems according to the embodiments. It will be understood that each block of the flowchart illustrations, and combinations of blocks in the flowchart illustrations, can be implemented by computer program instructions. These computer program instructions may be loaded onto a computer or other programmable apparatus to produce a machine, such that the instructions which execute on the computer or other programmable apparatus create means for implementing the functions specified in the flowchart block or blocks. These computer program instructions may also be stored in a computer-readable memory that can direct a computer or other programmable apparatus to function in a particular manner, such that the instructions stored in the computer-readable memory produce an article of manufacture including instruction means which implement the function specified in the flowchart block or blocks. The computer program instructions may also be loaded onto a computer or other programmable apparatus to cause a series of operational steps to be performed on the computer or other programmable apparatus to produce a computer programmable apparatus which provides steps for implementing the functions specified in the flowchart block or blocks.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An assist control apparatus, comprising:
a driving mechanism attached to a leg of a user;
an acquisition unit configured to acquire a status signal indicating a motion of an arm of the user;
an estimation unit configured to determine an assistance timing which is a timing for assisting an action of the user based on changes in the status signal; and
a drive unit configured to drive the driving mechanism to generate an assistance power to assist the action of the user in accordance with the assistance timing.

2. The apparatus according to claim 1, wherein the estimation unit estimates the action of the user based on the changes in the status signal.

3. The apparatus according to claim 1, further comprising:
a first calculation unit configured to calculate an assistance amount indicating a strength of the assistance power in accordance with the motion of the user and the assistance timing,
wherein the drive unit drives the driving mechanism to generate the assistance power corresponding to the assistance amount.

4. The apparatus according to claim 1, wherein the estimation unit determines whether to increase or decrease the assistance power based on the changes in the status signal.

5. The apparatus according to claim 1, wherein the driving mechanism is attached to the user so as to assist a movement of at least one of a waist, knee, and ankle of the user.

6. The apparatus according to claim 1, wherein the acquisition unit acquires, as the status signal, a biosignal including a myoelectric potential of the arm of the user.

7. The apparatus according to claim 1, wherein the acquisition unit acquires, as the status signal, a sensor signal including at least one of a position of the arm of the user, speed, and acceleration of movement of the arm of the user measured by a sensor.

8. The apparatus according to claim 1, wherein the estimation unit estimates, as the action of the user, a walking motion, standing-up motion, and sitting-down motion based on a positional relation between a body and the arm of the user.

9. The apparatus according to claim 1, further comprising:
a sensor attached to the leg of the user and configured to acquire a detection signal by measuring a speed and an acceleration of movement of the arm;
a second calculation unit configured to calculate a delayed time indicating a delay of movement of a lower limb of the user relative to movement of an upper limb of the user in a walking motion by using the status signal and the detection signal; and
a correction unit configured to correct the assistance timing to be advanced by the delayed time.

10. The apparatus according to claim 9, wherein the correction unit adjusts a driving timing so that a difference between the assistance timing and the driving timing is less than a first time period if the difference is no less than the first time period, the driving timing being a timing to drive the driving mechanism.

11. The apparatus according to claim 1, wherein the driving mechanism is attached to at least one of a right leg and a left leg of the user, and the acquisition unit acquires the status signal indicating a movement of one of right and left arms of the user.

12. An assist control method, comprising:
acquiring a status signal indicating a motion of an arm of the user;
determining an assistance timing which is a timing for assisting an action of the user based on changes in the status signal; and
driving a driving mechanism attached to a leg of the user to generate an assistance power to assist the action of the user in accordance with the assistance timing.

13. The method according to claim 12, further comprising estimating the action of the user based on the changes in the status signal.

14. The method according to claim 12, further comprising:
calculating an assistance amount indicating a strength of the assistance power in accordance with the motion of the user and the assistance timing,
wherein the driving drives the driving mechanism to generate the assistance power corresponding to the assistance amount.

15. The method according to claim 12, wherein the determining determines whether to increase or decrease the assistance power based on the changes in the status signal.

16. The method according to claim 12, wherein the driving mechanism is attached to the user so as to assist a movement of at least one of a waist, knee, and ankle of the user.

17. The method according to claim 12, wherein the acquiring acquires, as the status signal, a biosignal including a myoelectric potential of the arm of the user.

18. The method according to claim 12, wherein the acquiring acquires, as the status signal, a sensor signal including at least one of a position of the arm of the user, speed, and acceleration of movement of the arm of the user measured by a sensor.

19. The method according to claim 12, further comprising estimating, as the action of the user, a walking motion, standing-up motion, and sitting-down motion based on a positional relation between a body and the arm of the user.

20. The method according to claim 12, further comprising:
acquiring a detection signal by measuring a speed and an acceleration of movement of the arm by attaching a sensor to the leg of the user;
calculating a delayed time indicating a delay of movement of a lower limb of the user relative to movement of an upper limb of the user in a walking motion by using the status signal and the detection signal; and
correcting the assistance timing to be advanced by the delayed time.
